(19) United States
(12) Patent Application Publication
(10) Pub. No.: US 2026/0072004 A1
(43) Pub. Date: Mar. 12, 2026

(54) Floating Water Sensor Device with Tube Enclosed Antennae

(71) Applicant: Precision Measurement Engineering, Inc., Vista, CA (US)

(72) Inventors: Michael Jay Head, Encinitas, CA (US); Kristin Michelle Elliott, San Marcos, CA (US)

(21) Appl. No.: 19/325,137

(22) Filed: Sep. 10, 2025

Related U.S. Application Data
(60) Provisional application No. 63/693,290, filed on Sep. 11, 2024.

(51) Int. Cl. G01N 33/18; H01Q 1/34

(52) U.S. Cl. CPC G01N 33/1886; H01Q 1/34

(57) ABSTRACT

A water sensor buoy for use in a water environment includes an elongate tubular case with a case body with a case interior and a case exterior surface. A water sensor is in the case interior adjacent a sensor case end and exposed to the water environment. The water sensor senses an attribute of the water environment. A float is removably attached to the case exterior surface and positionable along the case body to offset the water sensor from the float. A data processing and control unit is in electronic communication with the water sensor to generate a data signal upon receipt of sensor data. An antennae supported by the tubular case in the case interior. A data transmission unit is in electronic communication with the data processing and control unit to wirelessly transmit a data signal generated by the data processing and control unit using the antennae.

### CROSS-REFERENCE TO RELATED APPLICATIONS

This application relates to and claims the benefit of U.S. Provisional Application No. 63/693,290 filed Sep. 11, 2024, and entitled “FLOATING WATER SENSOR WITH TUBE ENCLOSED ANTENNA” the entire disclosure of which is hereby wholly incorporated by reference.

### STATEMENT RE: FEDERALLY SPONSORED RESEARCH/DEVELOPMENT

Not Applicable

### BACKGROUND

There are a variety of attributes of water or aqueous environments that are desirable to be measured and monitored. Such water environments may range from oceans, lakes, reservoirs, holding ponds, to pools. Various sensors or probes may be deployed in such water environments. For example, optical fluorescence sensors are single wavelength in situ fluorescence and turbidity probes used for monitoring various water quality parameters such as chlorophyll-a concentration and turbidity in aquatic systems. Acoustic backscatter sensors are used to measure the concentration and size distribution of suspended particles or sediments in water. Photosynthetically active radiation (PAR) sensors work by measuring the intensity of light that is used by plants for photosynthesis. There are a variety of types of conductivity sensors available that are used for measuring the electrical conductivity of water, which is related to the concentration of dissolved materials in the water, such as salts. Hydrophones are underwater microphones that are used to detect and measure sound waves in water. There are a variety of pH sensors for measuring the acidity or alkalinity of a solution.

Such water sensors may be deployed in a mobile manner, such as installed in a float or buoy. These water sensor buoys would have a hull or housing that contains a sealable chamber or inflatable bladder for floatation on the water surface. The water sensor would typically be installed at the bottom side of the buoy to be exposed to the water environment when the buoy is floating. Sensor data may then be collected and processed by the water sensor and associated electronics which are housed within the buoy hull. In some applications, sensed data may also be wirelessly transmitted by the water sensor buoy so as to enable retrieval of real-time or current data. An antennae is usually affixed to the topside of the buoy hull or housing above the water line for receipt of GPS data to which may be correlated to the sensor data. An array of these water sensor buoys may be utilized for a desired coverage area.

In view of the foregoing, there is a need in the art for an improved water sensor buoys.

### BRIEF SUMMARY

According to an aspect of the invention, there is provided a floating water sensor device for use in a water environment. The water sensor device includes an elongate tubular case sized and configured to float in the water environment. The tubular case has a case body with a case interior and a case exterior surface. The case body has an antennae case end and a sensor case end. The water sensor device further includes a water sensor supported by the tubular case and disposed in the case interior adjacent the sensor case end. The water sensor is exposed to the water environment upon the water sensor device being placed in the water environment. The water sensor is sized and configured to sense an attribute of the water environment and generate sensor data based upon the sensed attribute. The water sensor device further includes a float removably attached to the case exterior surface and positionable along the case body to selectively offset the water sensor from the float. The float has an overall density less than water. The water sensor device further includes a data processing and control unit supported by the tubular case and disposed in the case interior. The data processing and control unit is in electronic communication with the water sensor and sized and configured to generate a data signal upon receipt of sensor data from the water sensor. The water sensor device further includes an antennae supported by the tubular case and completely disposed in the case interior. The antennae extends longitudinally towards the antennae case end. The water sensor device further includes a data transmission unit supported by the tubular case and disposed in the case interior. The data transmission unit is in electronic communication with the data processing and control unit. The data transmission unit is sized and configured to wirelessly transmit a data signal generated by the data processing and control unit using the antennae. The water sensor device further includes a power source supported by the tubular case and disposed in the case interior. The power source being in electrical communication with the data processing and control unit and the data transmission unit.

According to various embodiments, the tubular case may have an antennae end cap disposed at the antennae case end sized and configured to seal the case interior at the antennae case end. The tubular case may further have a sensor end cap disposed at the sensor case end sized and configured to seal the case interior at the sensor case end. The sensor end cap may have a sensor end cap opening. The water sensor may be exposed to the water environment upon the water sensor device being placed in the water environment through the sensor end cap opening. The float may have a float central opening and the case body is disposed through the float central opening. The water sensor device may further include a float retainer attached to the case exterior surface longitudinally between the float and the antennae case end for restricting the float from sliding toward the antennae case end. The antennae may have an antennae base and an antennae tip. The antennae is supported by the tubular case by the antennae base. The antennae tip longitudinally extends towards the antennae case end. The float is generally aligned along the case body longitudinally adjacent to the antennae base. The water sensor device may further include a ballast. The ballast is attached to the tubular case adjacent the sensor case end. The ballast has an overall density greater than water. The water sensor device may further include a ribbon cable sized and configured to electronically connect the data transmission unit to the data processing and control unit. The case body of the tubular case may have a round cross-sectional shape. The case body of the tubular case may have a same cross-sectional shape longitudinally along the length of the case body.

The present invention will be best understood by reference to the following detailed description when read in conjunction with the accompanying drawings.

### BRIEF DESCRIPTION OF THE DRAWINGS

These and other features and advantages of the various embodiments disclosed herein will be better understood with respect to the following description and drawings, and in which:

FIG. 1 is a top perspective view of a water sensor device according to an aspect of the present invention;

FIG. 2 is a bottom perspective view of the water sensor device of FIG. 1;

FIG. 3 is an exploded view of the water sensor device of FIG. 1;

FIG. 4 is an exploded view of a portion of the water sensor device of FIG. 3 including a tube case;

FIG. 5 is a side symbolic side view of the water sensor device of FIG. 1 as deployed in a water environment;

FIG. 6 is a cross-sectional side view of the water sensor device of FIG. 1 as viewed along axis 6-6 of FIG. 5;

FIG. 7 is an assembled top perspective view the portion of the water sensor device of FIG. 4 with the tube case depicted in phantom lining; and

FIG. 8 is a further exploded top perspective view of the portion of water sensor device of FIG. 7 without the tube case.

Common reference numerals are used throughout the drawings and the detailed description to indicate the same elements.

### DETAILED DESCRIPTION

The above description is given by way of example, and not limitation. Given the above disclosure, one skilled in the art could devise variations that are within the scope and spirit of the invention disclosed herein. Further, the various features of the embodiments disclosed herein can be used alone, or in varying combinations with each other and are not intended to be limited to the specific combination described herein. Thus, the scope of the claims is not to be limited by the illustrated embodiments. It is further understood that the use of relational terms such as top and bottom, first and second, and the like are used solely to distinguish one entity from another without necessarily requiring or implying any actual such relationship or order between such entities.

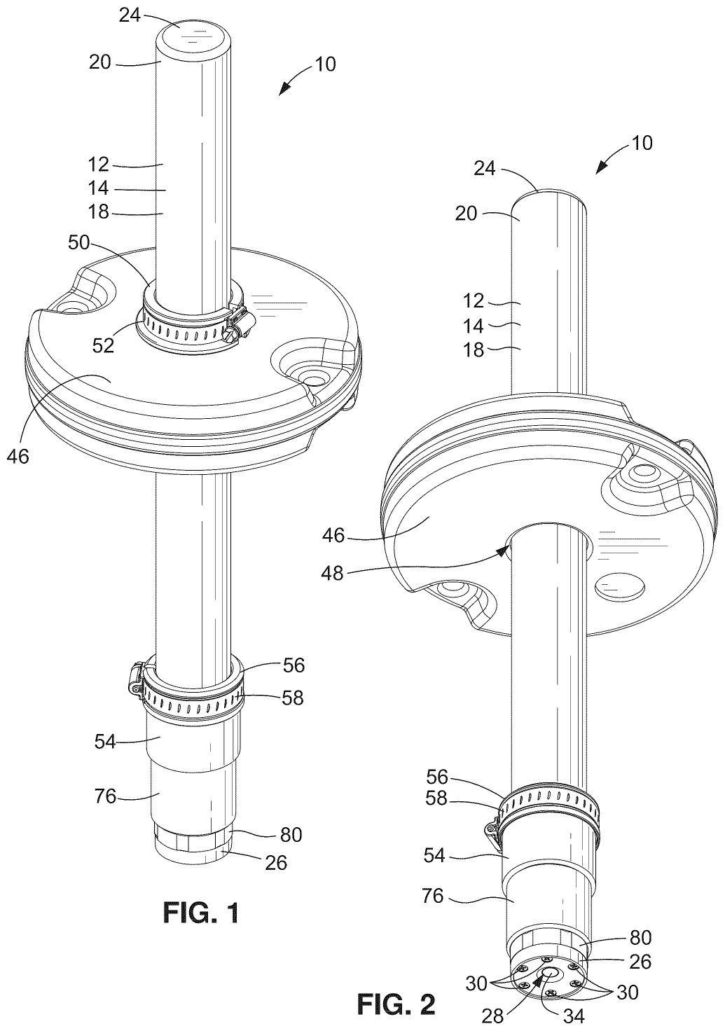

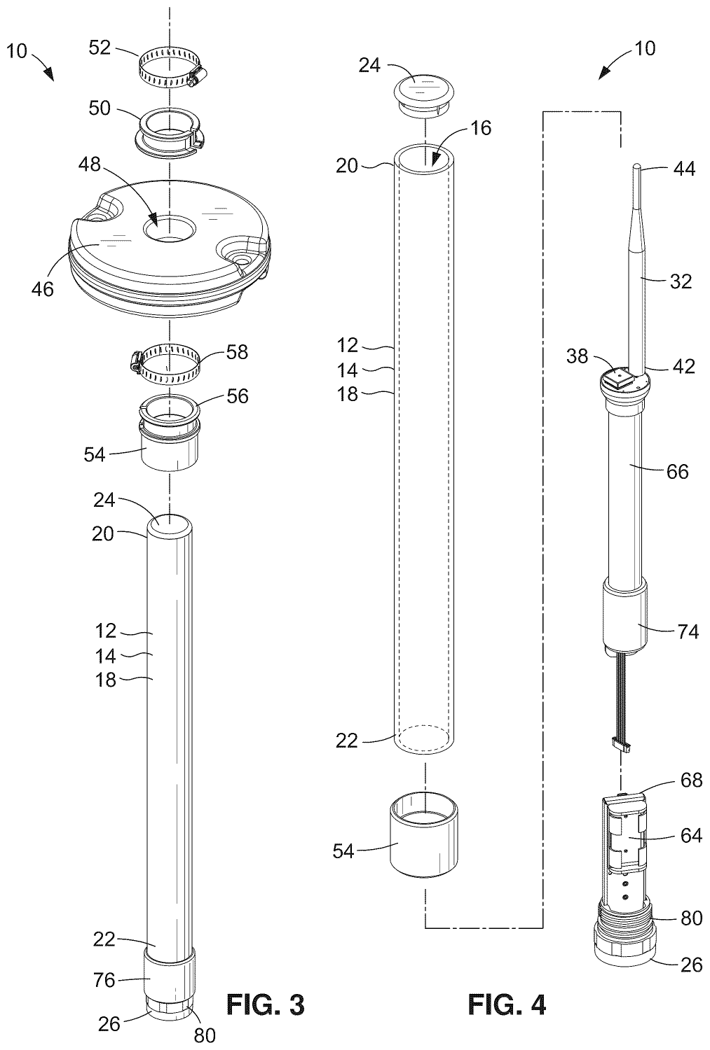

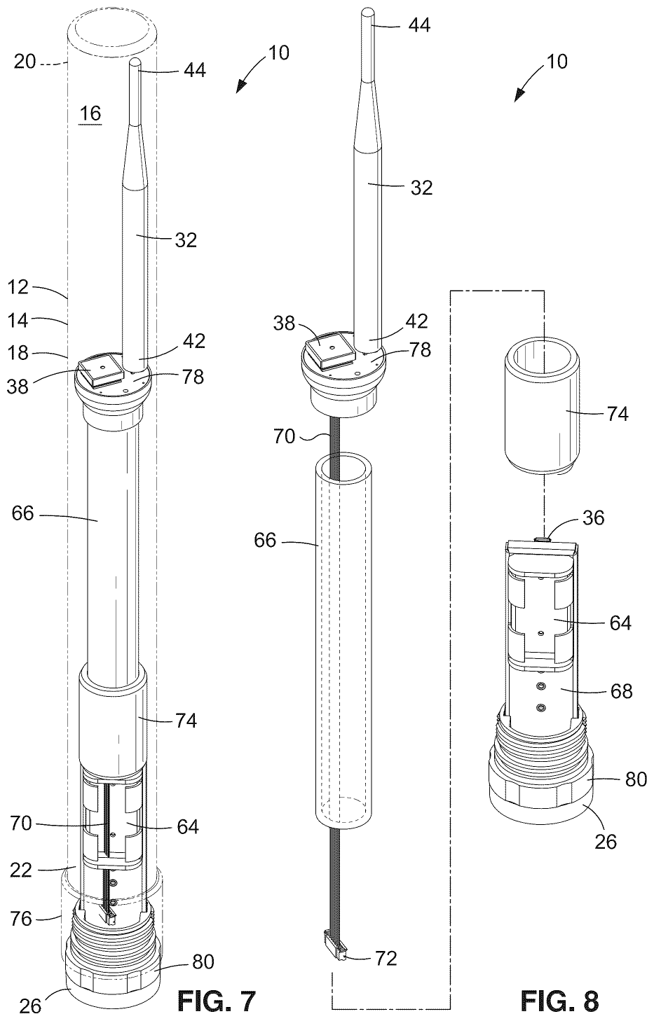

Referring now to FIGS. 1-8, according to an aspect of the present invention, there is provided a floating water sensor device 10 for use in a water environment 60. FIG. 1 is a top perspective view of the water sensor device 10, and FIG. 2 is a bottom perspective view of the water sensor device of FIG. 1. FIG. 3 is an exploded view of the water sensor device 10 of FIG. 1. The water sensor device 10 includes an elongate tubular case 12 sized and configured to float in the water environment 60. The tubular case 12 has a case body 14 with a case interior 16 and a case exterior surface 18. The case body 14 has an antennae case end 20 and a sensor case end 22. The water sensor device 10 further includes a water sensor 34 supported by the tubular case 12 and disposed in the case interior 16 adjacent the sensor case end 22. The water sensor 34 is exposed to the water environment 60 upon the water sensor device 10 being placed in the water environment 60. The water sensor 34 is sized and configured to sense an attribute of the water environment 60 and generate sensor data based upon the sensed attribute.

The water sensor device further includes a float 46 removably attached to the case exterior surface 18 and positionable along the case body 14 to selectively offset the water sensor 34 from the float 46. The float 46 has an overall density less than water. As such, the attachment of the float add a buoyancy for supporting the overall weight of water sensor device 10.

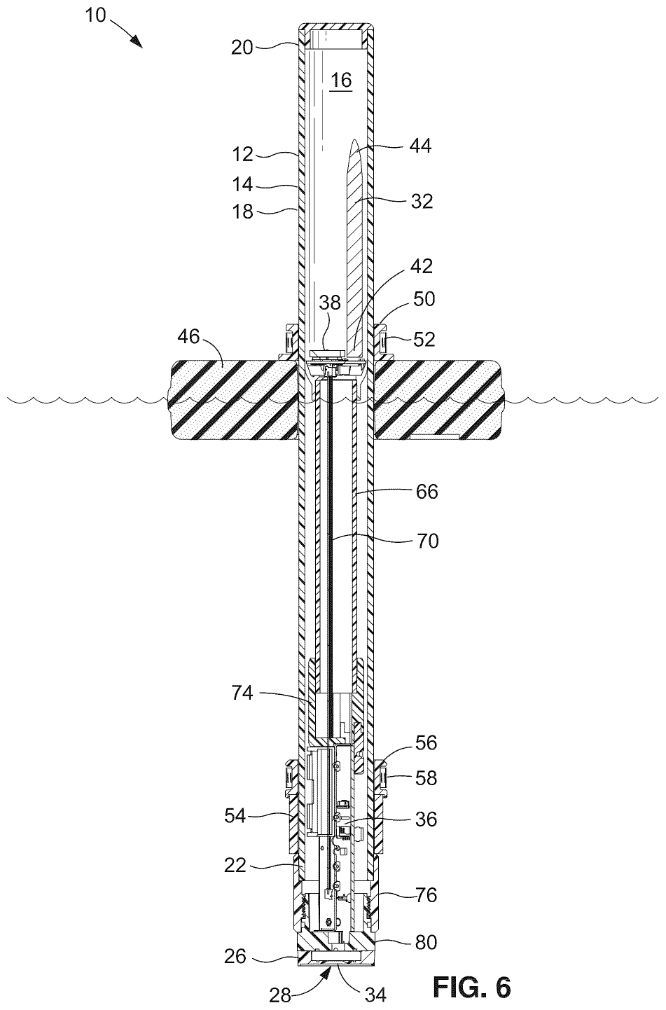

With additional reference to FIGS. 6-8, the water sensor device 34 further includes a data processing and control unit 36 supported by the tubular case 12 and disposed in the case interior 16. The data processing and control unit 36 is in electronic communication with the water sensor 34 and sized and configured to generate a data signal upon receipt of sensor data from the water sensor 34. The water sensor device 10 further includes an antennae 32 supported by the tubular case 12 and completely disposed in the case interior 16. The antennae 32 extends longitudinally towards the antennae case end 20. The water sensor device 10 further includes a data transmission unit 38 supported by the tubular case 12 and disposed in the case interior 16. The data transmission unit 38 is in electronic communication with the data processing and control unit 36. The data transmission unit 38 is sized and configured to wirelessly transmit a data signal generated by the data processing and control unit 36 using the antennae 32. The water sensor device 10 further includes a power source 40 supported by the tubular case 12 and disposed in the case interior 16. The power source 40 is in electrical communication with the data processing and control unit 36 and the data transmission unit 38.

The tubular case 12 is used as an overall floatation mechanism for the overall water sensor device 10. An aspect of the present invention is the utilization of the tubular case 12 that is used to house in a watertight environment all of the functional components of the water sensor 34 and associated electronics, namely, the antennae 32, the data processing and control unit 36, the data transmission unit 38, and the power source 40. The elongate nature of the tubular case 12 is particularly suitable for housing the antennae 32 which is itself is contemplated to be in an elongate form factor. It is contemplated that the antenna 32 (being installed in the housing in the tubular case 12) allows for the antenna to be conveniently protected from the elements. As such, the antenna 32 and related fittings and such do not need to be particularly weatherized, which is contemplated to be more expensive and still prone to corrosion.

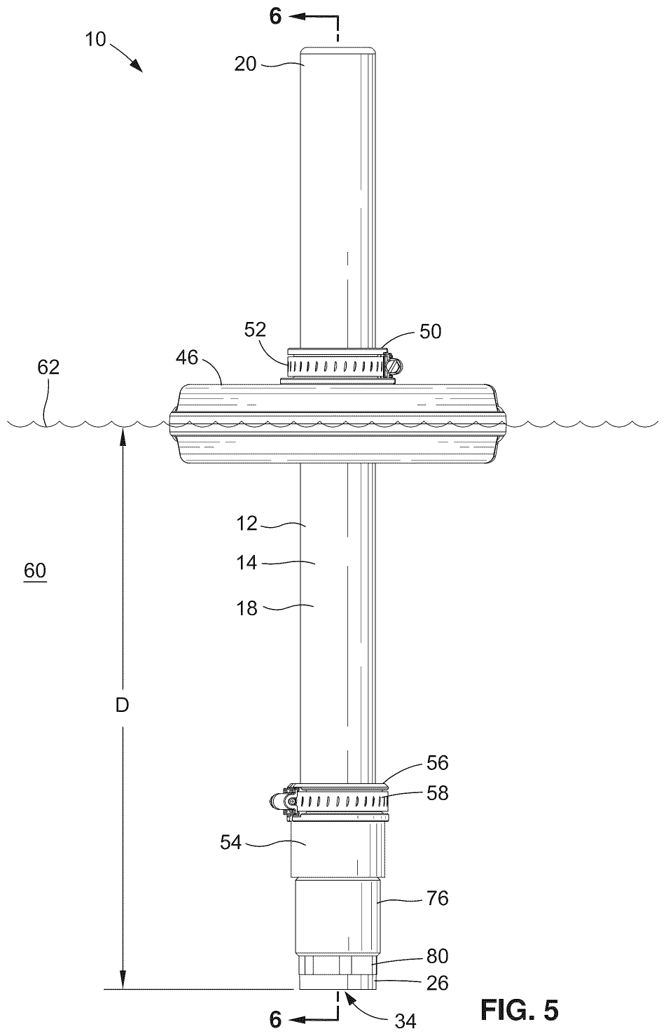

Significantly, the tube-like construction of the tubular case 12 engaged for an easy and efficient means of lengthening the entire assembly. In this regard, this design of the water sensor device 10 allows for usage of different tubular cases 12 with different lengths for an ease of scaling. Because this design provides for the water sensor 34 to be positioned at the sensor case end 20 that may be positioned at various depths in the water environment 60. A longer tubular case 12 allows the water sensor 34 to measure at various depths in the water environment 60. FIG. 5 is a side symbolic side view of the water sensor device 10 as deployed in a water environment 60. The water depth “D” denotes the depth/distance between the water sensor 34 and a waterline 62. Various tubular cases 12 may be readily manufactured as the tubular case 12 is formed of a case body 14 that may be formed of relatively low cost tubing materials which are easily cut to different lengths. The case body 14 may have a round cross-sectional shape, and the case body 14 may have a same cross-sectional shape longitudinally along the length of the case body 14.

This is important as water attributes and parameters can differ depending on the depth and measuring at different depths is necessary to fully understand the characteristics of the water environment 60 in which water sensor device 10e is deployed. Having the ability to “customize” the length of the case body 14 and in turn the measurement depth is significant for this device as there are many different applications which require measurements at different depths. For example, shrimp farms need to measure at the specific depth of their tanks where the shrimp largely reside. Alternatively, fish ponds need to be monitored at multiple depths due to much more significant depth of the pond and the stratification of the water.

According to various embodiments, the tubular case 12 may have an antennae end cap 24 disposed at the antennae case end 20 sized and configured to seal the case interior 16 at the antennae case end 20. In this respect the antennae end cap 24 may be glued, fused or otherwise in sealed engagement with the case body 14 at the antennae case end 20 so as to be watertight.

The tubular case 12 may further have a sensor end cap 26 disposed at the sensor case end 22 sized and configured to seal the case interior 16 at the sensor case end 22. However, the sensor end cap 26 may be removeably attached to the sensor case end 22 so as to allow for the various electronic components of the water sensor device 10 to be assembled into the tubular case 12 to be assembled into the tubular case 12 and later removed as desired. Fasteners 30 are used to secure the sensor end cap 26 in the embodiment depicted. As such, with this design the maintaining of the watertight environment within the tubular case 12 just needs to focus on ensuring a watertight seal at the sensor case end 22. As such, the fully enclosed device design of the water sensor device 10 embodies an advantage in that it only has one seal at the bottom. The reduced number of seals on a water sensor apparatus reduces the required maintenance and risk of leaks.

The sensor end cap 26 may be attached to or integrated with an assembly mount 80. The assembly mount 80 may be configured to engage a tube connector 76. In the embodiment depicted, the assembly mount 80 includes outward facing threads which are configured to engage with inward facing threads at a threaded end of the tube connector 76. This threaded engagement is contemplated to be a removably watertight engagement. The tube connector 76 on the opposite end from the threaded end is tube engagement end that this sized and configured to engage the case body 14 by receiving the case body in a collar-like engagement. This may be with a press-fit engagement with additional sealing materials, like a silicon glue or other arrangement chosen from those which are well known to one of ordinary skill in the art. With this configuration, the case body 14 may be formed of a mere tubing material without modification allowing its manufacture to be as simple as cutting to designed length.

A circuit board support 64 may be provided that is attached to the assembly mount 80. The water sensor 34 may be attached to the assembly mount 80 adjacent to the sensor end cap. The power source 64 may be attached to the assembly mount 80. A circuit board support 68 may may also be attached to the assembly mount 80. The data processing and control unit 36 may be mounted to the circuit board support 80.

An antennae spacer 66 may be provided that is attached to the circuit board support 68 through the use of a spacer connector 74. The spacer connector 74 may be configured to receive the circuit board support 68 at one end and the antennae spacer 66 at the other end. The spacer connector 74 extends from the circuit board support 68 towards the antennae case end 20. The spacer connector 74 may take the form of a length of tubing. The antenna 32 may have an antennae base 42 and an opposing antennae tip 44. The antennae 32 may be mounted to an antennae support 78 at the antennae base 42. The antennae support 78 may be attached to the end of the antennae spacer 66. The data transmission unit 38 may also be attached to the antennae support 78 conveniently adjacent to the antennae 32. A ribbon cable 70 may be connected to the data transmission unit 38 and extend along and within the antennae spacer 66. The ribbon cable 70 may terminate at a cable connector 72 that is configured to electronically connect with the data processing and control unit 36. It is contemplated that additional electronic components may be mounted on the antennae support 78, such as additional antennae (such as may be dedicated for receiving data signals, like GPS signals).

With this configuration, the offset distance of the antennae 32 from the water sensor 34 may be readily accomplished by providing an antennae spacer 66 of an appropriate length. As mentioned above, the antennae spacer 66 may be formed of tubing which is low cost material that may be customized by simply cutting tubing to the desired length. The length of the ribbon cable 70 to be provided so as to be a suitably long enough length so as to accommodate a variety of anticipated offset distances between the antennae 32 and the water sensor 34.

The float 46 may be generally aligned along the case body 14 longitudinally adjacent to the antennae base 42. The float 46 supplies supplementary floatation and buoyancy to ensure the top portion of the water sensor device 10 where the antenna 32 is located is above the water line 62. This is necessary to ensure the water does not block or interfere with the function of the receipt and/or transmission of signals via the antennae 32. The float 46 may have a float central opening 48 and the case body 14 is disposed through the float central opening 48. The water sensor device 10 may further include a float retainer 50 attached to the case exterior surface 18 longitudinally between the float 46 and the antennae case end 20 for restricting the float 46 from sliding toward the antennae case end 20. A float retainer clamp 52 may be used to affix the float retainer 50 to the case body 14. The overall design and configuration of the float 46 may be chosen from those which are well known to one of ordinary skill in the art. It is understood that the particular embodiment depicted of the float 46 is exemplary in nature. Other floatation arrangements may be utilized that incorporate various materials, such as foam components, and use of inflatable air bladders for example.

The water sensor device may further include a ballast 54. The ballast 54 is attached to the tubular case 12 adjacent the sensor case end 22. A ballast retainer 56 may be provided that is attached about the case body 14 so as to prevent the ballast 54 from sliding along the case body 14 towards the float 46. A ballast retainer clamp 58 may be used to affix the ballast retainer 56 to the case body 14. The ballast 54 has an overall density greater than water. The ballast 54 is used to ensure that the lower portion of the water sensor device 10 maintains the vertical configuration of the sensor case end 22 being below the antennae case end 20.

As mentioned above, the water sensor 34 is sized and configured to sense an attribute of the water environment 12 and generate sensor data for receipt by the data processing and control unit 36. The water sensor 34 may be any number of sensors used to detect or sense a physical property of the water or liquid in which the water sensor device 10 is deployed. The water sensor 34 may be chosen from those which are well known to one of ordinary skill in the art. For example, the water sensor 34 may be optical fluorescence sensors, acoustic backscatter sensors, photosynthetically active radiation (PAR) sensors, conductivity sensors, hydrophones, and pH sensors. Some general descriptions are below.

Optical fluorescence sensors are single wavelength in situ fluorescence and turbidity probes used for monitoring various water quality parameters such as chlorophyll-a concentration and turbidity in aquatic systems. The probes consist of a light source and a detector that measure the intensity of the emitted fluorescence and scattered light, respectively. The probe is submerged in the water and emits light at a specific wavelength through a clear face, such as the exterior sensor surface 36, which excites chlorophyll-a and other fluorescent compounds in the water. The emitted fluorescence is then detected by a detector of the probe through the same clear face and measured in terms of its intensity. Turbidity, which is a measure of the amount of suspended particles in the water, is measured by the detector of the probe through the detection of scattered light. The data obtained from these probes can be used for various environmental monitoring applications, such as assessing the health of aquatic ecosystems, detecting harmful algal blooms, and monitoring water treatment processes.

Acoustic backscatter sensors are used to measure the concentration and size distribution of suspended particles or sediments in water. These sensors work by emitting an acoustic signal, typically in the range of 1 to 10 MHz, and measuring the echo or backscatter of the signal as it interacts with particles in the water. These sensors typically consist of a transducer that emits the acoustic signal and receives the backscatter of the signal, and a signal processing unit that converts the received signal into data on the concentration and size distribution of the particles. The transducer emits a short pulse of sound waves that travel through the water and interact with the particles. Some of the sound waves are scattered back toward the transducer, and these echoes are received and processed by the signal processing unit. The strength of the backscatter signal is related to the concentration and size of the particles in the water. Relatively larger and denser particles will produce a stronger backscatter signal than smaller and less dense particles. By analyzing the backscatter signal, the sensor can be used to determine the concentration and size distribution of particles in the water. Acoustic backscatter sensors are used in a variety of applications, such as monitoring sediment transport in rivers and coastal environments, assessing the quality of drinking water, and studying the behavior of plankton and other suspended particles in the ocean or other water environment.

Photosynthetically active radiation (PAR) sensors work by measuring the intensity of light in the wavelength range of 400 to 700 nanometers, which corresponds to the range of light that is used by plants for photosynthesis. These sensors typically consist of a photodiode or photovoltaic cell that is sensitive to light in this range and a signal processing unit that converts the light intensity into an electrical signal that can be recorded or analyzed. When light hits the photodiode or photovoltaic cell in the sensor, it generates a small electric current that is proportional to the intensity of the light. The signal processing unit amplifies the electric current and converts it into a voltage signal that can be recorded or transmitted to other devices for further analysis.

There are a variety of types of conductivity sensors available that are used for measuring the electrical conductivity of water, which is related to the concentration of dissolved materials in the water, such as salts. These sensors include inductive conductivity sensors, four-electrode conductivity sensors, contacting conductivity sensors, toroidal conductivity sensors and optical conductivity sensors. Inductive conductivity sensors measure the electrical conductivity of water using an inductive coil and an electrode system. The inductive coil generates a magnetic field, which induces an electrical current in the water. The current is then measured by the electrode system, which is proportional to the conductivity of the water. Four-electrode conductivity sensors measure the conductivity of water using four electrodes. Two of the electrodes are used to apply an AC current to the water, while the other two electrodes measure the voltage drop across the water. By measuring the voltage drop, the conductivity of the water can be calculated. Contacting conductivity sensors measure the conductivity of water using two electrodes that are in contact with the water. The electrical resistance between the electrodes is measured, and the conductivity of the water is calculated using Ohm's Law. Toroidal conductivity sensors measure the conductivity of water using a toroidal coil that surrounds the water. The coil generates a magnetic field, which induces an electrical current in the water. The current is then measured, and the conductivity of the water is calculated. Optical conductivity sensors measure the conductivity of water using an optical method, where light is passed through the water and the absorption of the light is measured. The absorption is related to the conductivity of the water.

Hydrophones are underwater microphones that are used to detect and measure sound waves in water. These sensor work by converting sound waves into electrical signals that can be recorded or analyzed. Hydrophones consist of a piezoelectric element that is housed in a cylindrical or spherical case. The piezoelectric element is made of a material that generates an electrical signal when subjected to pressure or vibration. When sound waves travel through water, they create variations in pressure that cause the piezoelectric element to vibrate. The vibrations generate electrical signals that are proportional to the sound wave's amplitude and frequency. Hydrophones can be used for a wide range of applications, such as oceanography, marine biology, underwater communications, and military sonar systems. The sensitivity and frequency response of a hydrophone depend on several factors, such as the size and shape of the piezoelectric element, the acoustic properties of the surrounding water, and the design of the hydrophone housing.

There are a variety of pH sensors for measuring the acidity or alkalinity of a solution. These include glass electrode pH sensors, ion-selective field-effect transistor (ISFET) pH sensors, optical pH sensors, conductivity-based pH sensors, and microelectromechanical system (MEMS) pH sensors. Glass electrode pH sensors are the most common type of pH sensors. They consist of a glass membrane that is sensitive to changes in pH and an electrode that measures the potential difference between the sample solution and a reference solution. Ion-selective field-effect transistor (ISFET) pH sensors use a thin film transistor and a gate electrode that is sensitive to changes in pH. The gate electrode is covered with a pH-sensitive material that interacts with the sample solution, causing a change in the transistor's electrical properties. Optical pH sensors use fluorescent dyes or indicators that change their optical properties in response to changes in pH. The changes in the fluorescence can be measured and used to determine the pH of the sample solution. Conductivity-based pH sensors use the principle of conductivity to measure changes in pH. The conductivity of a solution is affected by changes in pH, and the changes in conductivity can be used to determine the pH of the solution. Microelectromechanical system (MEMS) pH sensors use microfabrication techniques to create tiny mechanical structures that can measure changes in pH. The mechanical structures are coated with a pH-sensitive material, and changes in the mechanical properties can be used to determine the pH of the sample solution. Overall, each type of pH sensor has its own advantages and limitations, and the choice of sensor depends on the specific application and the accuracy required for the measurement.

The sensor end cap 26 may have a sensor end cap opening 28. The water sensor 34 may be exposed to the water environment 60 upon the water sensor device 10 being placed in the water environment 60 through the sensor end cap opening 28. In this respect an actual physical contact of a portion of the water sensor 34 may be necessary or merely viewable thereat (such as in the case of an optical type sensor) depending on the nature of the particular type of water sensor 34 being deployed. As mentioned above, the water sensor 34 is exposed to the water environment 60 upon the water sensor device 10 being placed in the water environment 60. It is understood that only the sensing element, such as a sensor foil, would be exposed to the water environment 60, whereas a remainer of the water sensor 10 (such as electronic components and wiring would not). The water sensor 34 may be provided by a sensor manufacturer or vendor. However, depending upon the nature of the particular sensor type, it is contemplated that additional protective lenses or layer, such as an additional glass layer or pane. The water sensor 26 may include a sensor foil that is exposed at the sensor end cap opening 28. Additional lenses or protective covers may additionally be utilized. Furthermore, the structures and methods for effecting a suitable watertight seal for the water sensor 34 at the sensor end cap opening 28 to only expose the water sensor 34 as needed without allowing any leakage may be chosen from those which are well known to one of ordinary skill in the art.

The water sensor 26, the antennae 32, the data processing and control unit 52, the data transmission unit 54, the power source 56 and the associated circuitry, wiring and interconnections may be chosen from any of those designs and arrangements which are well known in one of ordinary skill in the art. While the various components are depicted as being separate components it is understood that various ones of the components may be combined in a single unit. In addition, it is contemplated that the water sensor device 10 may include other electrical components such as GPS location hardware/software and other or additional water sensors or environmental sensors (such as for measuring water temperature, air temperature and pressure, surface wind, etc.).

The data processing and control unit 36 is configured to process the sensor data or data signals generated by the water sensor 34 as may be desired for transmission by the data transmission unit 38. The data processing and control unit 36 generally is configured to control the operation of the other on-board electronics with data and control signals as well as power distribution. The data processing and control unit 36 may feature other functions, such as running system diagnostics for generating error/fault/status data associated with any of the on-board electronics.

The particulars shown herein are by way of example only for purposes of illustrative discussion, and are presented in the cause of providing what is believed to be the most useful and readily understood description of the principles and conceptual aspects of the various embodiments set forth in the present disclosure. In this regard, no attempt is made to show any more detail than is necessary for a fundamental understanding of the different features of the various embodiments, the description taken with the drawings making apparent to those skilled in the art how these may be implemented in practice.

What is claimed is:

1. A floating water sensor device for use in a water environment, the water sensor device comprising:
an elongate tubular case sized and configured to float in the water environment; tubular case having case body with a case interior and a case exterior surface, the case body having an antennae case end and a sensor case end;
a water sensor supported by the tubular case and disposed in the case interior adjacent the sensor case end, the water sensor being exposed to the water environment upon the water sensor device being placed in the water environment, the water sensor being sized and configured to sense an attribute of the water environment and generate sensor data based upon the sensed attribute;
a float removably attached to the case exterior surface and positionable along the case body to selectively offset the water sensor from the float, the float having an overall density less than water;
a data processing and control unit supported by the tubular case and disposed in the case interior, the data processing and control unit being in electronic communication with the water sensor and sized and configured to generate a data signal upon receipt of sensor data from the water sensor;
an antennae supported by the tubular case and completely disposed in the case interior, the antennae extending longitudinally towards the antennae case end;
a data transmission unit supported by the tubular case and disposed in the case interior, the data transmission unit being in electronic communication with the data processing and control unit, the data transmission unit being sized and configured to wirelessly transmit a data signal generated by the data processing and control unit using the antennae; and
a power source supported by the tubular case and disposed in the case interior, the power source being in electrical communication with the data processing and control unit and the data transmission unit.

2. The water sensor device of claim 1 wherein the tubular case has an antennae end cap disposed at the antennae case end sized and configured to seal the case interior at the antennae case end.

3. The water sensor device of claim 2 wherein the tubular case further has a sensor end cap disposed at the sensor case end sized and configured to seal the case interior at the sensor case end, the sensor end cap having a sensor end cap opening, the water sensor being exposed to the water environment upon the water sensor device being placed in the water environment through the sensor end cap opening.

4. The water sensor device of claim 1 wherein the float has a float central opening, the case body is disposed through the float central opening.

5. The water sensor device of claim 4 further includes a float retainer attached to the case exterior surface longitudinally between the float and the antennae case end for restricting the float from sliding toward the antennae case end.

6. The water sensor device of claim 1 wherein the antennae has an antennae base and an antennae tip, the antennae is supported by the tubular case by the antennae base, the antennae tip longitudinally extends towards the antennae case end.

7. The water sensor device of claim 6 wherein the float is generally aligned longitudinally along the case body adjacent to the antennae base.

8. The water sensor device of claim 1 further includes a ballast, the ballast is attached to the tubular case adjacent the sensor case end, the ballast having an overall density greater than water.

9. The water sensor device of claim 1 further includes a ribbon cable sized and configured to electronically connect the data transmission unit to the data processing and control unit.

10. The water sensor device of claim 1 wherein the case body of the tubular case has a round cross-sectional shape.

11. The water sensor device of claim 1 wherein the case body of the tubular case has a same cross-sectional shape longitudinally along the length of the case body.
